(19) United States
(12) Patent Application Publication
(10) Pub. No.: US 2005/0197713 A1
(43) Pub. Date: Sep. 8, 2005

(54) Ternary single-phase ceramic medical devices

(76) Inventor: Mark G. Catlin, Arden Hills, MN (US)

Correspondence Address: MERCHANT & GOULD PC, P.O. BOX 2903, MINNEAPOLIS, MN, 55402-0903, US

(21) Appl. No.: 11/068,059

(22) Filed: Feb. 28, 2005

Related U.S. Application Data
(60) Provisional application No. 60/549,247, filed on Mar. 1, 2004.

(51) Int. Cl. A61F 2/28

(52) U.S. Cl. 623/23.56; 501/87; 501/96.1

(57) ABSTRACT

The invention relates to medical devices made from ternary single-phase ceramics. In an embodiment, the invention is a medical orthopedic implant comprising at least a portion made of a single-phase ceramic with a formula of Mₙ₊₁AXₙ (wherein n=1 to 3, M is an early transition metal, A is an A-group element, and X is C and/or N). In an embodiment, the invention is a method of treating a disorder associated with orthopedic malformation and/or dysfunction and wherein an implantation of artificial implant is needed, in a subject in need of such treatment, the method comprising implanting in said subject an implant having at least a portion thereof made of a single-phase ceramic with a formula of Mₙ₊₁AXₙ (wherein n=1 to 3, M is an early transition metal, A is an A-group element, and X is C and/or N.

### CROSS REFERENCE TO RELATED APPLICATION

This application claims the benefit of U.S. Provisional Patent Application Ser. No. 60/549,247, filed Mar. 1, 2004, which application is hereby incorporated by reference in its entirety.

### FIELD OF THE INVENTION

The invention is related to medical devices. More specifically, the invention is related to prosthetic medical devices.

### BACKGROUND OF THE INVENTION

Trauma, degenerative joint disease, autoimmune diseases, metabolic diseases, hereditary deformity and other processes often result in painful, restricted joints. Often the pain and limitations become intolerable and joint replacement is sought. In the year 2000, over 1.6 million joint replacement procedures were performed, mostly for arthritis.

There has been a continual quest to improve the materials from which these prosthetic devices are made. The gold, silver, wood and bone of antiquity have been replaced by advanced metal alloys, plastics and ceramics. However, perfection has not been achieved and current devices are still plagued by failure resulting in activity restrictions, a limited implant life span and ultimately the need for replacement of the prosthetic device.

Therefore, a need exists for medical devices made from a more suitable material.

### SUMMARY OF THE INVENTION

The invention relates to medical devices made from ternary single-phase ceramics. In an embodiment, the invention is a medical orthopedic implant comprising at least a portion made of a single-phase ceramic with a formula of Mₙ₊₁AXₙ (wherein n=1 to 3, M is an early transition metal, A is an A-group element, and X is C and/or N). In an embodiment, the invention is a method of treating a disorder associated with orthopedic malformation and/or dysfunction wherein an implantation of artificial implant is needed. The method comprises implanting in a subject an implant having at least a portion thereof made of a single-phase ceramic with a formula of Mₙ₊₁AXₙ (wherein n=1 to 3, M is an early transition metal, A is an A-group element, and X is C and/or N).

The above summary of the present invention is not intended to describe each discussed embodiment of the present invention. This is the purpose of the figures and the detailed description that follows.

### DRAWINGS

The invention may be more completely understood in connection with the following drawings, in which:

FIG. 1 shows diagrams of the crystal structures of ternary single-phase ceramics.

FIG. 2 is a cross-sectional view of the crystal structure of a ternary single-phase ceramic.

FIG. 3 is a view of a hip implant as it is installed in a patient in accordance with an embodiment of the invention.

FIG. 4 is an exploded view of the hip implant of FIG. 3.

FIG. 5 is a view of a knee implant as it is installed in a patient in accordance with an embodiment of the invention.

FIG. 6 is an exploded view of the knee implant of FIG. 5.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The invention relates to medical devices made from ternary single-phase ceramics. In an embodiment, the invention is a medical orthopedic implant comprising at least a portion made of a single-phase ceramic with a formula of Mₙ₊₁AXₙ (wherein n=1 to 3, M is an early transition metal, A is an A-group element, and X is C and/or N). In an embodiment, the invention is a method of treating a disorder associated with orthopedic malformation and/or dysfunction and wherein an implantation of artificial implant is needed, in a subject in need of such treatment, the method comprising implanting in said subject an implant having at least a portion thereof made of a single-phase ceramic with a formula of Mₙ₊₁AXₙ (wherein n=1 to 3, M is an early transition metal, A is an A-group element, and X is C and/or N).

Materials:

Orthopedic implant materials should preferably combine high strength, low friction, mechanical energy absorption, corrosion resistance, heat conduction, tissue compatibility, and minimum wear particle generation.

In an embodiment, materials of the invention include ceramics with the general formula of Mn+1AXₙ (wherein n=1 to 3, M is an early transition metal, A is an A-group element, and X is C and/or N). Exemplary early transition metals include Sc, Ti, V, Cr, Zr, Nb, Mo, Hf, and Ta. Exemplary A-group elements include Al, Si, P, S, Ga, Ge, As, Cd, In, Sn, Tl, and Pb. Where n=1, the resulting compound is known as a 211 compound. Exemplary 211 compounds include Ti₂AlC, Ti₂AlN, Hf₂PbC, Cr₂GaC, V₂AsC, Ti₂InN, Nb₂AlC, (Nb,Ti)₂AlC, Ti₂AlN_{0.5}C_{0.5}, Nb₂GaC, Nb₂AsC, Zr₂InN, Ti₂GeC, Cr₂AlC, Zr₂SC, Mo₂GaC, Ti₂CdC, Hf₂InN, Zr₂SnC, Ta₂AlC, Ti₂SC, Ta₂GaC, Sc₂lnC, Hf₂SnN, Hf₂SnC, V₂AIC, Nb₂SC, Ti₂GaN, Ti₂InC, Ti₂TlC, Ti₂SnC, V₂PC, Hf₂SC, Cr₂GaN, Zr₂InC, Zr₂TlC, Nb₂SnC, Nb₂PC, Ti₂GaC, V₂GaN, Nb₂InC, Hf₂TlC, Zr₂PbC, Ti₂PbC, V₂GaC, V₂GeC, Hf₂InC, Zr₂TlN. Where n=2, the resulting compound is known as a 312 compound. Exemplary 312 compounds include Ti₃AlC₂, Ti₃GeC₂, and Ti₃SiC₂. Where n=3, the resulting compound is known as a 413 compound. Suitable 413 compounds include Ti₄AlN₃. By way of example, U.S. Pat. No. 5,942,455, the disclosure of which is herein incorporated by reference, describes various methods of synthesizing 312 compounds.

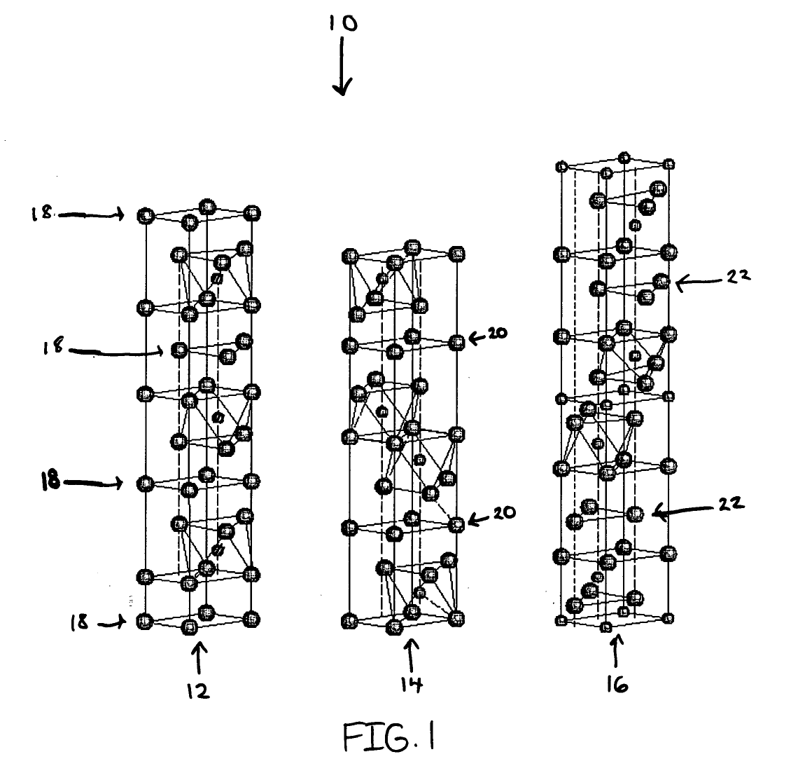

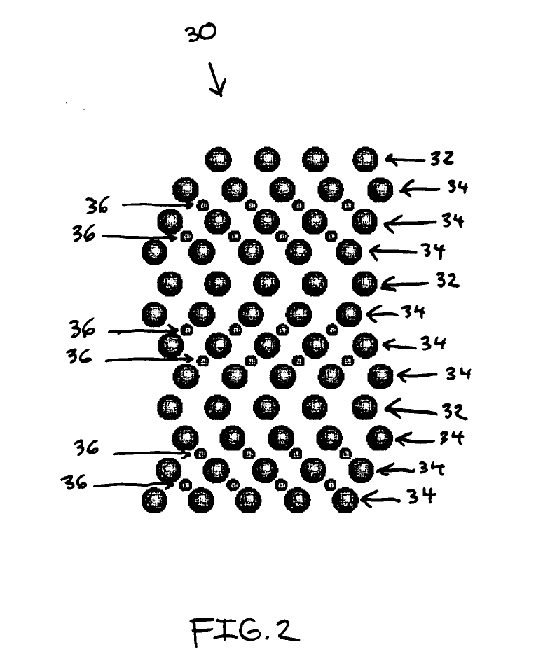

Such ternary compounds can form single phase ceramics with properties including high strength, low friction, mechanical energy absorption (compressibility), oxidation resistance, heat conduction, tissue compatibility, minimum wear particle generation, and machinability. These materials are also damage tolerant. The material has a laminar structure. Referring now to FIG. 1, diagrams 10 of the crystal structures of ternary single-phase ceramics are shown. For a 211 compound, the A-group elements 18 comprise every third layer. For a 312 compound, the A-group elements comprise every fourth layer. For a 413 compound, the A-group elements comprise every fifth layer. Referring now to FIG. 2, a cross-sectional view of the crystal structure of a 312 compound is shown. In this view, the relation of the A-group atoms 32 are shown in relation to C or N atoms 36 and the early transition metal (M) 34 atoms.

In typical ceramics, extreme deformation creates kink boundaries that resist crack extension. Although not self-healing, damage tolerance is built in at the atomic level. The result is that this material is unlikely to catastrophically fail.

In some embodiments, the ternary single-phase ceramics are compressible so they can mechanically absorb and dissipate energy. In has been reported that these ceramics can dissipate up to 25% of the mechanical energy applied per cycle. The energy dissipated is more significant with coarse-grained material. Similar to natural cartilage, such materials can absorb the energy of high impacts generated during running and jumping.

The compressibility can be varied. For example, the compressibility can be custom-tuned for each application by variation of the grain size and orientation during the fabrication process. A different formulation can be made for different implants based on mechanical requirements. A hip acetabular cup may require different characteristics than the tibial surface. The characteristics of the material may be varied depending on the patients weight and expected activities.

Surface Coatings and Treatments:

In some embodiments, a layer or coating of a different material may be disposed on the surface of the ternary single-phase ceramic of the invention. By way of example, the surface can be heat-treated with a graphite foil, creating titanium carbide, one of the hardest materials known.

In some embodiments, a layer of a ternary single-phase ceramic is applied in a layer on the exterior of a medical device that may be the same ternary single-phase ceramic that comprises the joint or may be a different ternary single-phase ceramic. By way of example, Ti₃SiC₂ can be thermally sprayed, either by high-velocity oxy-fuel in air or plasma in vacuum, onto substrates with relative ease. Alternatively such materials can be sprayed onto a metallic substrate, such as steel. The sprayed coatings are resistant to corrosion by most acids.

In some embodiments, the surface of the ternary single-phase ceramic is polished. In an embodiment, the surface of the ternary single-phase ceramic is hardened. For example, the implant-implant contact surfaces can be hardened. Once machined to the tolerances required, the surfaces that would tend to wear, such as the implant-implant contact surfaces would be treated to increase carbon content to enhance wear resistance. Alternatively, to prevent oxidation, the surfaces of the ternary single-phase ceramic could have their silicon content raised to enhance their oxidation resistance and increase biocompatibility. In an embodiment, the bone contact surfaces may be textured or treated to facilitate bone ingrowth.

Medical Devices:

The ternary single-phase ceramics described above can be used in making medical implants, prostheses, joints, etc. combination of high strength and light weight means potentially smaller prosthetics with less bone revision to fit the implants. Medical devices that can be made in accordance with embodiments of the invention include a permanent endo-prosthesis and a permanent exo-prosthesis.

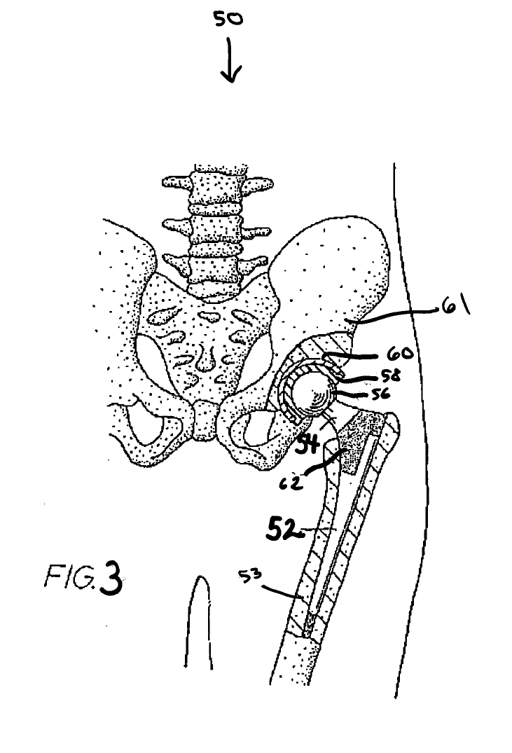

In an embodiment of the invention, ternary single-phase ceramics are used to form the parts of a hip joint. Referring now to FIG. 3, a view of a hip implant 50 is shown as it is installed in a patient in accordance with an embodiment of the invention. The hip joint stem 52 fits into the femur 53 while the femoral head 56 of the prosthesis fits into and articulates against the inner lining 58 of an acetabular cup 60 which in turn is affixed to the pelvis 61.

In an embodiment, the hip joint stem fully comprises a ternary single-phase ceramic material. In an embodiment, the hip joint stem partially comprises a ternary single-phase ceramic material. In an embodiment, the hip joint stem is coated with a ternary single-phase ceramic material. In an embodiment, the femoral head of the prosthesis fully comprises a ternary single-phase ceramic material. In an embodiment, the femoral head of the prosthesis partially comprises a ternary single-phase ceramic material. In an embodiment, the femoral head is coated with a ternary single-phase ceramic material. In an embodiment, the inner lining of the acetabular cup fully comprises a ternary single-phase ceramic material. In an embodiment, the inner lining of the acetabular cup partially comprises a ternary single-phase ceramic material. In an embodiment, the inner lining of the acetabular cup is coated with a ternary single-phase ceramic material. In an embodiment, the acetabular cup fully comprises a ternary single-phase ceramic material. In an embodiment, the acetabular cup partially comprises a ternary single-phase ceramic material. In an embodiment, the acetabular cup is coated with a ternary single-phase ceramic material.

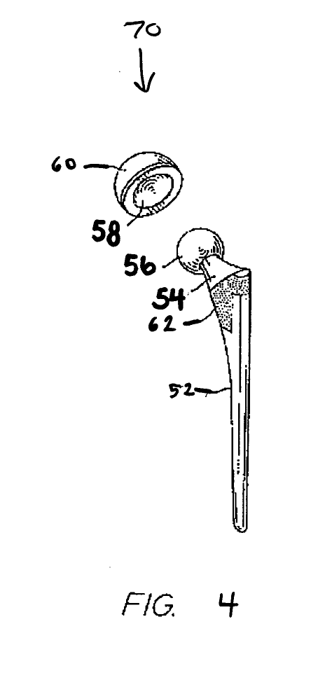

In some embodiments, a porous metal bead or wire mesh coating 62 may be incorporated to allow stabilization of the implant by in-growth of surrounding tissue into the porous coating. Similarly, such a coating can also be applied to the acetabular component. The femoral head 56 may be an integral part of the hip joint stem 52 or may be a separate component mounted upon a conical taper at the end of the neck 54 of the hip joint prosthesis. This allows the fabrication of a prosthesis having separate properties for the stem and femoral head. Referring now to FIG. 4, an exploded view 70 of the hip implant of FIG. 3 is shown. In this view, the inner lining 58 of an acetabular cup 60 is more clearly shown.

In an embodiment of the present invention, both the femoral stem and head fully comprise ternary single phase ceramics. In an embodiment of the present invention, both the femoral stem and head partially comprise ternary single phase ceramics. In a particular embodiment, both the femoral stem and head comprise titanium silicon carbide. In an embodiment, the surface of the femoral head is treated to form titanium carbide. In some embodiments, the surface is polished. In some embodiments, a wire mesh or metal bead made of titanium silicon carbide coats the stem to promote in-growth of tissue or bone into the device, thereby stabilizing its position. In an embodiment of the invention, the acetabular cup comprises a ternary single-phase ceramic.

In some embodiments, each component has unique physical characteristics. For example, surface hardness and smoothness are may be important for the femoral head. In an embodiment, the femoral head is has great surface hardness and smoothness. Toughness and fracture resistance may be important in the stem. In an embodiment, the stem is tough and fracture resistant. Compressibility, surface smoothness and fracture resistance need to be optimized in the acetabular cup. In an embodiment, the acetabular cup is compressible, has surface smoothness and is fracture resistant.

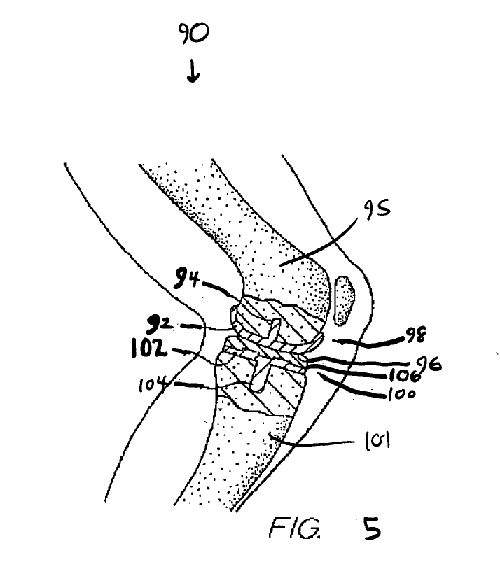

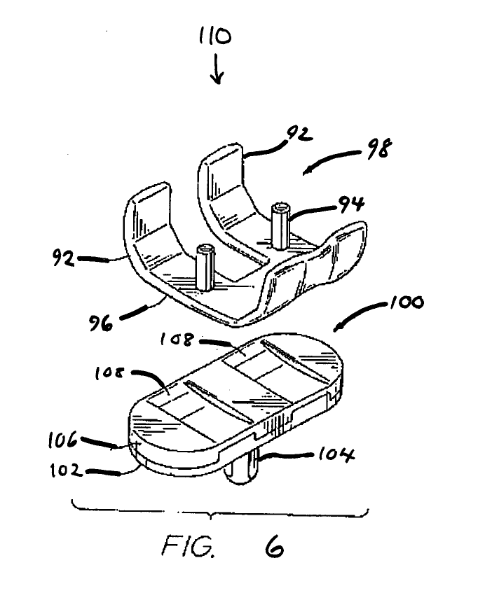

In an embodiment of the invention, ternary single-phase ceramics are used to form the parts of a knee prosthesis. Referring now to FIG. 5, a knee prosthesis 90 in accordance with an embodiment of the invention is shown. A knee prosthesis 90 consists of femoral 98 and tibial 100 components. In an embodiment the femoral and tibial components fully comprise a ternary single-phase ceramic material. In an embodiment the femoral and tibial components partially comprise a ternary single-phase ceramic material. In an embodiment the femoral and tibial components are coated with a ternary single-phase ceramic material. The upper or femoral component 98 has an implant portion 94 for inserting into the femur 95 of a patient, and a bearing surface having at least one condyle 92 on the prosthesis body. In an embodiment the implant portion fully comprises a ternary single-phase ceramic material. In an embodiment the implant portion partially comprises a ternary single-phase ceramic material. In an embodiment the implant portion is coated with a ternary single-phase ceramic material. In an embodiment, the condyle fully comprises a ternary single-phase ceramic material. In an embodiment, the condyle partially comprises a ternary single-phase ceramic material. In an embodiment, the condyle is coated with a ternary single-phase ceramic material. The tibial component 100, in most prostheses, has a tibial insert 106 that attaches to an underlying tibial tray 102. In an embodiment, the tibial insert fully comprises a ternary single-phase ceramic material. In an embodiment, the tibial insert partially comprises a ternary single-phase ceramic material. In an embodiment, the tibial insert is coated with a ternary single-phase ceramic material. In an embodiment, the tibial tray fully comprises a ternary single-phase ceramic material. In an embodiment, the tibial tray partially comprises a ternary single-phase ceramic material. In an embodiment, the tibial tray is coated with a ternary single-phase ceramic material. The tibial insert 106 surface is shaped with grooves 108 (shown in FIG. 6) to conform to the bottom surface 96 of the femoral condyle 92. The tibial tray 102 generally has a post 104 or some other means of fixing to the tibia 101. In an embodiment, the tibial insert and the tibial tray are integral and comprise a ternary single-phase ceramic material.

Knee prostheses typically are of two varieties: fixed bearing knees (FBK) and mobile bearing knees (MBK). Referring to FIG. 6, the interface in the FBK 110 is fixed and the only movement is between the top of the tibial component 100 and the condyles 92. In contrast, the MBK (not shown) is characterized by having its articulating tibial component movable relative to the tibial platform. As a result, the MBK has a greater range of motion but suffers from greater wear and creep than an FBK.

In an embodiment of the present invention, the femoral component, the tibial insert and tibial tray all comprise a ternary single-phase ceramic. In a specific embodiment, the femoral component, the tibial insert and tibial tray all comprise titanium silicon carbide. In an embodiment, only the tibial tray comprises a ternary single-phase ceramic.

In an embodiment, the femoral component is compressible. In an embodiment, the surface of the femoral component is hardened and polished. The tibial insert maybe of a greater or lesser thickness. In an embodiment, the tibial insert is compressible. In an embodiment, the surface of the tibial insert is hardened and polished.

In some embodiments, the tibial insert is capable of resisting sheer forces when the femoral condyles impact and twist. In an embodiment, the undersurface is hardened to resist torque forces at the interlock with the tibial tray.

In an embodiment, beads and wire mesh are applied to surfaces embedded in or in contact with the patient's femur or tibia. In some embodiments, the prosthetics of the invention may be sterile-packaged.

In an embodiment of the invention, ternary single-phase ceramics may be used to form prosthetics of many types. In an embodiment, ternary single-phase ceramics may be used to form both joints lacking a joint cavity and synovial joints. Joints of the invention may include a ball and socket joint, a hinge joint, a pivot joint, an ellipsoidal joint, a saddle joint and a gliding joint. By way of example, joints of the invention can include a hip joint, a knee joint, an ankle joint, a shoulder joint, an elbow joint, a wrist joint, a finger joint, a finger metacarpal joint, a toe joint, a toe-metatarsal joint, a carpo-metacarpal joint, and elements of the spine.

In some embodiments of the invention, ternary single-phase ceramics are used to form prosthetics which cover and overlie a patient's natural joint. By way of example, a hip cap may be formed of a ternary single-phase ceramic which does not replace the patient's hip joint but merely serves to line the natural hip joint.

While the present invention has been described with reference to several particular implementations, those skilled in the art will recognize that many changes may be made hereto without departing from the spirit and scope of the present invention.

1. A medical orthopedic implant comprising at least a portion made of a single-phase ceramic with a formula of Mₙ₊₁AXₙ (wherein n=1 to 3, M is an early transition metal, A is an A-group element, and X is C and/or N).

2. The medical implant of claim 1, wherein the entire medical implant is made of said ceramic.

3. The medical implant of claim 1, wherein the medical implant is an orthopedic implant.

4. The medical implant of claim 3, wherein the medical implant is selected from the group consisting of: a permanent endo-prosthesis and a permanent exo-prosthesis.

5. The medical implant of claim 3, wherein the medical implant is a joint replacement prosthesis.

6. The medical implant of claim 1, further comprising a portion made of at least one other material.

7. The medical implant of claim 6, wherein said at least one other material is selected from the group consisting of polymers, ceramics, metals and materials coated with a substance to enhance bone ingrowth.

8. A method of treating a disorder associated with orthopedic malformation and/or dysfunction and wherein an implantation of artificial implant is needed, in a subject in need of such treatment, the method comprising implanting in said subject an implant having at least a portion thereof made of a single-phase ceramic with a formula of Mₙ₊₁AXₙ (wherein n=1 to 3, M is an early transition metal, A is an A-group element, and X is C and/or N).

9. The process of claim 8, wherein the entire medical implant is made of said ceramic.

10. A method of treating a damaged or degenerative arthroplasty associated with malformation and/or dysfunction of a joint, and wherein an implantation of an artificial joint implant is needed, in a subject in need of such treatment, the method comprising implanting in said subject an artificial joint implant made of a single-phase ceramic with a formula of Mₙ₊₁AXₙ (wherein n=1 to 3, M is an early transition metal, A is an A-group element, and X is C and/or N).

11. The process of claim 10, wherein the entire medical implant is made of said ceramic.

12. The method of claim 10, wherein said joint is selected from the group consisting of joints lacking a joint cavity and a synovial joint.

13. The method of claim 12 wherein said synovial joint is selected from the group consisting of: a ball and socket joint, a hinge joint, a pivot joint, an ellipsoidal joint, a saddle joint and a gliding joint.

14. The method of claim 12, wherein said synovial joint is selected from the group consisting of a hip joint, a knee joint, an ankle joint, a shoulder joint, an elbow joint, a wrist joint, a finger joint, a finger metacarpal joint, a toe joint, a toe-metatarsal joint and a carpo-metacarpal joint.

15. A medical implant made of a material other than a single-phase ceramic with a formula of Mₙ₊₁AXₙ (wherein n=1 to 3, M is an early transition metal, A is an A-group element, and X is C and/or N), said medical implant being coated with a single-phase ceramic with a formula of Mₙ₊₁AXₙ (wherein n=1 to 3, M is an early transition metal, A is an A-group element, and X is C and/or N).

16. A medical implant comprising at least a portion made of a single-phase ceramic with a formula of Mₙ₊₁AXₙ (wherein n=1 to 3, M is an early transition metal, A is an A-group element, and X is C and/or N), said medical implant being sterile-packaged.
